(19) United States
(12) Patent Application Publication
(10) Pub. No.: US 2021/0095351 A1
(43) Pub. Date: Apr. 1, 2021

(54) Methods of Preparing Dual Indexed Methyl-Seq Libraries

(71) Applicant: INTEGRATED DNA TECHNOLOGIES, INC., Coralville, IA (US)

(72) Inventors: Ushati Das Chakravarty, San Mateo, CA (US); Hsiao-Yun Huang, Fremont, CA (US); Yu Zheng, Lexington, MA (US); Kevin Lai, Fremont, CA (US)

(21) Appl. No.: 17/036,986

(22) Filed: Sep. 29, 2020

Related U.S. Application Data
(60) Provisional application No. 62/907,778, filed on Sep. 30, 2019.

(51) Int. Cl. C12Q 1/6888; C12N 15/10

(52) U.S. Cl. CPC C12Q 1/6888; C12Q 2600/154; C12N 15/1093

(57) ABSTRACT

The invention pertains the methods and compositions for generating methyl-seq NGS libraries, for whole genome sequencing or targeted resequencing. Additionally, the invention pertains the methods and compositions for determining methylation profiles of target nucleic acids.

### CROSS REFERENCE TO RELATED APPLICATIONS

This application claims priority to U.S. Provisional Patent Application No. 62/907,778 filed Sep. 30, 2019, the contents of which are incorporated by reference herein in their entirety.

### FIELD OF THE INVENTION

The present invention pertains to methods for determining the sequence of double stranded DNA molecules and for the identification and profiling of methylated cytosine in double stranded DNA molecules. The invention also pertains to methods for constructing duplex consensus enabled next generation sequencing (NGS) methyl-seq libraries for whole genome sequencing, targeted resequencing, sequencing-based screening assays, metagenomics, or any other application requiring sample preparation for NGS.

### BACKGROUND OF THE INVENTION

DNA methylation is an epigenetic modification which is directly implicated in gene expression and chromatic structure regulation. Epigenetic modification, e.g., DNA methylation plays a role in mammalian development, for example, embryonic development, and is involved in chromatic structure and chromatin stability. Aberrant DNA methylation is implicated in a number of diseases processes, including cancer. Additionally, specific patterns of differentially methylated regions and/or allele specific methylation can be used as a molecule marker for non-invasive diagnostics. Importantly, methylation-focused whole-genome deep sequencing has revealed rich complexity in cancer methylomes, including hemimethylation or methylation on only one strand of the DNA duplex. Analysis of DNA methylation status across a genome or circulating cell-free DNA can be of interest.

Methods for profiling DNA methylation rely on bisulfite conversion sequencing.

Bisulfite treatment converts unmethylated cytosine residues into uracil. Once sequenced by Sanger sequencing or current NGS methods the uracil residues are visualized as thymine. On the other hand, methylcytosines are protected from conversion by bisulfite treatment to uracil. Once sequenced by Sanger sequencing or current NGS methods the methylcytosines are visualized as cytosine. Following bisulfite conversion or enzymatic conversion the conversion status of individual cytosine residues can be inferred by comparing the sequence to unmodified reference sequences.

However, current methods often introduce amplification or sequencing artifacts during library preparation and/or sequencing. These errors can negatively impact results of the DNA methylation analysis. Additionally, current methods do not provide the users with the ability to use Unique Molecular Identifiers (UMIs) during data analysis and distinguish between hemimethylated, fully methylated, and unmethylated events. Current methods rely on conversion of unmethylated cytosine to uracil prior to the attachment of adapters. Because conversion occurs prior to adapter addition it is impossible to distinguish hemimethylation events. Current methods do not provide for both whole genome methylation profiling and targeted sequencing methylation profiling. Therefore, there is a need in the art for a method that provides a comprehensive target capture system for regions where methylation is critical for gene expression.

Additionally, there is a need in the art for methods and compositions that permit accurate detection of methylation states with single base resolution and the detection of fully methylated and hemimethylated DNA.

### BRIEF SUMMARY OF THE INVENTION

Disclosed herein are methods and compositions for preparing dual index nucleic acid libraries for methylation profiling. Further the methods and compositions disclosed herein may rely on either bisulfite or enzymatic conversion of unmethylated cytosine. In various embodiments the disclosed methods and compositions use a two-step tagging process to tag target nucleic acids with UMIs prior to bisulfite treatment or enzymatic conversion of unmethylated cytosine present in the target sequence. The tagging process may add a single UMI to one strand or UMIs to each strand of the target nucleic acid. Following the tagging methods, the target nucleic acid is bisulfite treated or enzymatically treated to covert unmethylated cytosine to uracil. The UMIs are used to identify individual DNA molecules and reduce amplification or sequencing introduced artifacts increasing the accuracy of the DNA methylation analysis. Additionally, tagging each strand individually with a UMI prior to bisulfite treatment or enzymatic conversion enables error correction for direct comparison between hemimethylated, fully methylated and unmethylated events.

In one embodiment (FIG. 1A), the workflow for whole genome methyl-seq library construction is provided. Strand-specific molecular indexes (Unique Molecular Identifiers, UMIs) are attached to biological templates via blunt ligation followed by a gap-fill ligation reaction. In the first step, fragmented gDNA, FFPE DNA, or unsheared cfDNA is subjected to an end-repair reaction producing blunt 5′ phosphorylated inserts with free 3′ OH ends. Following end-repair, the first sequencing adaptor (for example, P7 for Illumina platforms) is attached to the 3′ end of insert DNA via blunt ligation using a T4 DNA ligase; one strand of the adaptor is 5′ adenylated to facilitate ligation, while the complementary strand is blocked on the 3′ end with dideoxy-A, dideoxy-T, dideoxy-C, or dideoxy G to prevent ligation (FIGS. 1A and 1B). The dC bases in the adapter are changed to methyl-dC to retain their original identity during downstream bisulfite treatment/enzymatic cytosine to uracil conversion. The second sequencing adaptor is then attached to the 5′ ends of biological inserts through a gap fill ligation reaction linking the 3′ ends of adaptor molecules to the phosphorylated 5′ ends of the inserts. The dC bases in the adapter are changed to methyl-dC to retain their original identity during downstream bisulfite treatment/enzymatic conversion. During the gap-fill ligation, complementary UMI bases are polymerized using TaqIT polymerase and a dNTP mix with dATP, dTTP, dGTP and methyl-dCTP. Following the second ligation, unmethylated cytosine is converted to uracil by bisulfite treatment or enzymatic treatment. The newly constructed library molecules can then be PCR amplified with an uracil compatible DNA polymerase to add sample barcodes. During this step, the uracil in the insert (target strand) is converted (polymerized) to thymine on the newly synthesized complementary strand. The resultant library is ready for whole genome bisulfite sequencing (WGBS) on an appropriate sequencing system, for example, but not limited to an Illumina platform.

In an alternate embodiment (FIG. 1B), the workflow for targeted methyl-seq library construction is provided. Strand-specific molecular indexes (Unique Molecular Identifiers, UMIs) are attached to biological templates via blunt ligation followed by gap-fill ligation reactions. In the first step, fragmented gDNA, FFPE DNA or unsheared cfDNA is subjected to an end-repair reaction producing blunt 5′ phosphorylated inserts with free 3′ OH ends. Following end-repair, the first sequencing adaptor (for example, P7 for Illumina platforms) is attached to the 3′ end of insert DNA via blunt ligation using a T4 DNA ligase; one strand of the adaptor is 5′ adenylated to facilitate ligation, while the complementary strand is blocked on the 3′ end with dideoxy-A, T, C, or G to prevent ligation (FIGS. 1A and 1B). The dC bases in the adapter are changed to methyl-dC to retain their original identity during downstream bisulfite treatment/enzymatic conversion. The second sequencing adaptor is then attached to the 5′ ends of biological inserts through a gap fill ligation reaction linking the 3′ ends of adaptor molecules to the phosphorylated 5′ ends of the inserts. The dC bases in the adapter are changed to methyl-dC to retain their original identity during downstream bisulfite treatment/enzymatic conversion. During the gap-fill ligation, complementary UMI bases are polymerized by TaqIT polymerase using a dNTP mix with dATP, dTTP, dGTP and methyl-dCTP. The target region of interest in the genome is enriched by hybridization capture using a custom panel of biotinylated probes. Following the target enrichment, unmethylated cytosine is converted by bisulfite or enzymatic treatment to uracil. The captured library molecules can then be PCR amplified with an uracil compatible DNA polymerase to add sample barcodes. During this step, the uracil in the insert (target strand) are converted (polymerized) to thymine on the newly synthesized complementary strand. The resultant library is ready for targeted sequencing on an appropriate sequencing platform, for example, but not limited to an Illumina platform.

### BRIEF DESCRIPTION OF THE DRAWINGS

FIG. 1A shows whole genome methyl-seq library construction workflow.

FIG. 1B shows targeted methyl-seq library construction workflow.

FIG. 2 demonstrates that that methyl-dCTP can be incorporated at similar efficiencies as compared to dCTP.

FIG. 3 demonstrates the detection of methylation by whole genome bisulfite sequencing.

FIG. 4 demonstrates the detection of methylation status when converting unmethylated cytosine to uracil using enzymatic conversion methods.

FIG. 5 demonstrates the detection of methylation status using targeted sequencing methods.

FIG. 6 demonstrates the probe design for hybridization capture methods and corresponding capture at 100 ng and 250 ng input amounts.

FIGS. 7A and 7B demonstrate that accurate methylation levels are identified from a low quantify 10 ng input sample and with reduced bias.

FIGS. 8A, 8B, and 8C demonstrate WGBS using low input cfDNA isolated from healthy samples and diseased samples.

FIGS. 9A, 9B, 9C, and 9D demonstrate targeted methyl-seq using custom epigenetics panels with standard tiling or 2× tiling.

### DETAILED DESCRIPTION OF THE INVENTION

The methods and compositions disclosed herein provide compositions and methods for preparing methyl-seq next generation sequencing libraries. Disclosed herein are methods of preparing indexed nucleic acid libraries for methylation profiling. Conversion of unmethylated cytosine of the target nucleic acid are converted to uracil with either bisulfite conversion or cytidine deaminases. In various embodiments, the methods use a two-step process to tag the target nucleic acid with unique molecular identifiers (UMI), wherein a first UMI is ligated to the 3′ end of the target nucleic acid. Optionally a second UMI may be added or ligated to the 5′ end of the target nucleic acid. Following addition of the adapters to the target nucleic acid the tagged nucleic acids are treated chemically or enzymatically to convert the unmethylated cytosine to uracil. The use of UMI and conversion following UMI addition reduce or substantially eliminate sequencing and/or amplification induced artifacts and improve the accuracy of the methylation analysis. Additionally, the conversion of unmethylated cytosine to uracil following adapter addition can be used to identify fully methylated (i.e., methylation events on both strands of the target nucleic acid), hemimethylated (i.e., methylation occurring on one strand of the double stranded target nucleic events) or unmethylated target nucleic acid. These and other advantages of the invention, as well as additional inventive features, will be apparent from the description of the invention provided herein.

In one embodiment a method of determining a methylation profile of a target nucleic acid is provided. The method comprises: a) obtaining the target nucleic acid; b) ligating a first adapter to the 3′ end of the target nucleic acid with a first ligase; c) ligating a second adapter to the 5′ end of the target nucleic acid with a second ligase to generate an adapter-target-adapter complex; d) converting unmethylated cytosine to uracil in the adapter-target-adapter complex to generate a converted target; e) optionally PCR amplifying the converted target; f) sequencing the converted target; g) comparing the sequence of the converted target to a reference sequence to determine the methylation profile of the target nucleic acid.

In an additional embodiment the target nucleic acid molecules are DNA. In a further embodiment the DNA is whole genomic DNA, cell free DNA (cfDNA), or formalin fixed paraffin embedded DNA (FFPE DNA).

In another embodiment the first ligase is a T4 DNA ligase. In a further embodiment the T4 DNA ligase is a mutant ligase. In another embodiment the mutant ligases contains an amino acid substitution at K159. In another embodiment the mutant ligase contains an amino acid substitution and is a K159S mutant.

In another embodiment the first or second adapter contains a unique molecular identifier sequence. In another embodiment the first and second adapter both contain a unique molecular identifier sequence.

In one embodiment the conversion of unmethylated cytosine to uracil is performed with bisulfite treatment. In another embodiment the conversion of unmethylated cytosine to uracil is performed with a cytidine deaminase.

In another embodiment the adapters comprise a universal priming site. In another embodiment following ligation of the adapters to form an adapter-target-adapter complex the complex is enriched by hybridization capture. The method of claim 1, wherein the adapter-target-adapter complex is enriched by hybridization capture.

In one embodiment a method for identifying methylated cytosine in a population of nucleic acids is provided. In further embodiments the nucleic acid is DNA and additionally the DNA is double stranded. In one embodiment, the methods of the invention are used for profiling the methylation pattern of whole genome, cfDNA, ctDNA, or FFPE DNA. The method in the described embodiments ensures sequence fidelity and increases the quality of the sequencing data. The methods in the described embodiments may comprise sequencing and identifying each strand of the double stranded DNA. Additionally, the methods in the described embodiments permit the identification of fully methylated and hemimethylated target nucleic acid and permits the distinction between fully methylated, hemimethylated, and unmethylated events in the target nucleic acid.

In addition, the invention provides for the generation of libraries and the sequencing of methylated target nucleic acid wherein the adapters used are barcoded or contain unique molecular identifiers. The use of UMI allows tracking of either strand of the double stranded target nucleic acid, that is the UMIs allow tracking of the sense or antisense strand of the original target nucleic acid. In one embodiment the UMIs are random. In another embodiment the UMI is rationally or intelligently designed, that is the UMI is designed such that the barcode is a known sequence. The UMI can be used to reduce amplification bias, which is the asymmetric amplification of different targets due to differences in nucleic acid composition. The UMI can be used to discriminate between nucleic acid mutations that arise during library preparation or during amplification, and mutations that were induced by bisulfite or enzymatic conversion of unmethylated cytosines to uracil. In some embodiments the UMIs can be greater than 2, 3, 4, 5, 6, 7, 8, 9, 10, 11, 12, 13, 14, 15, 16 ,17, 18, 19, or 20 nucleotides.

In another embodiment sample indexes or sample ID tags may be incorporated into the adapter. The sample index can be any suitable length from 2 to 18, from 3 to 18, from 4 to 18, from 5 to 18, from 6 to 18, from 7 to 18 or from 8 to 18 nucleotides in length. The sample ID tags can be of any length necessary to identify at least 2, at least 4, at least 256, at least 1024, at least 4096, or at least 16,384 or more individual samples.

In another embodiment universal priming sites may be incorporated into the adapter. The universal priming sites allow amplification of samples that have been tagged. Samples may be tagged by a UMI, by a sample ID, or a combination of UMI or sample ID.

In another embodiment conversion of the unmethylated cytosine to uracil can be accomplished with bisulfite treatment or with enzymatic treatment. In some embodiments the enzymatic treatment may be with a cytidine deaminase enzyme. In further embodiments the cytidine deaminase may be APOBEC. In some embodiments the cytidine deaminase includes activation induced cytidine deaminase (AID) and apolipoprotein B mRNA editing enzymes, catalytic polypeptide-like (APOBEC). In some embodiments, the APOBEC enzyme is selected from the human APOBEC family consisting of: APOBEC-1 (Apo1), APOBEC-2 (Apo2), AID, APOBEC-3A, -3B, -3C, -3DE, -3F, -3G, -3H and APOBEC-4 (Apo4). In some embodiments the conversion, whether by bisulfite conversion or enzymatic conversion, uses commercially available kits. In one example a kit such as EZ DNA Methylation-Gold, EX DNA Methylation-Direct or an EZ DNA Methylation-Lighting kit (available from ZYmo Research Corp (Irvine, California.)) is used. In another example a kit such as APOBEC-Seq (NEBiolabs) is used.

In another embodiment the adapters are added prior to conversion of the unmethylated cytosine to uracil. In a further embodiment the adapters contain UMIs. Adding adapters prior to conversion of the unmethylated cytosine to uracil allows the tracking of individual strands and permits the detection and profiling of fully methylated or hemimethylated events.

In another embodiment the adapter contains unmethylated cytosine. In yet another embodiment the adapter may contain unmethylated and methylated cytosine. In a further embodiment the adapter may contain all methylated cytosine. The dC bases in the adapter are changed to methyl-dC to retain their original identity during downstream bisulfite treatment/enzymatic cytosine to uracil conversion

The invention relates to a method for identifying methylated cytosine in a population of double stranded target nucleic acid. The double stranded target nucleic acid may be DNA. In further embodiments the DNA may be genomic DNA, sheared DNA, fragmented DNA, cfDNA, or FFPE DNA. In some embodiments the DNA may be end repaired and A-tailed or end repaired and blunted. In some embodiments the DNA is isolated from a biological sample for detection, diagnosis, or screening for a disease or disorder. In certain embodiments the biological sample may be tissue or tumor cells.

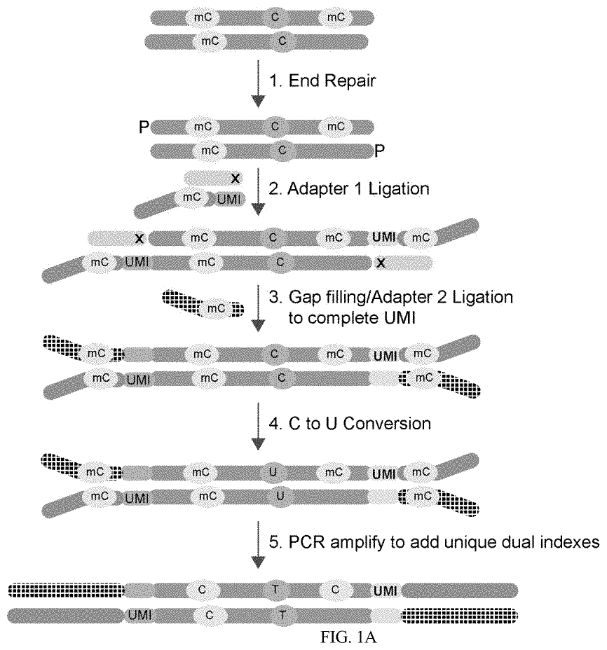

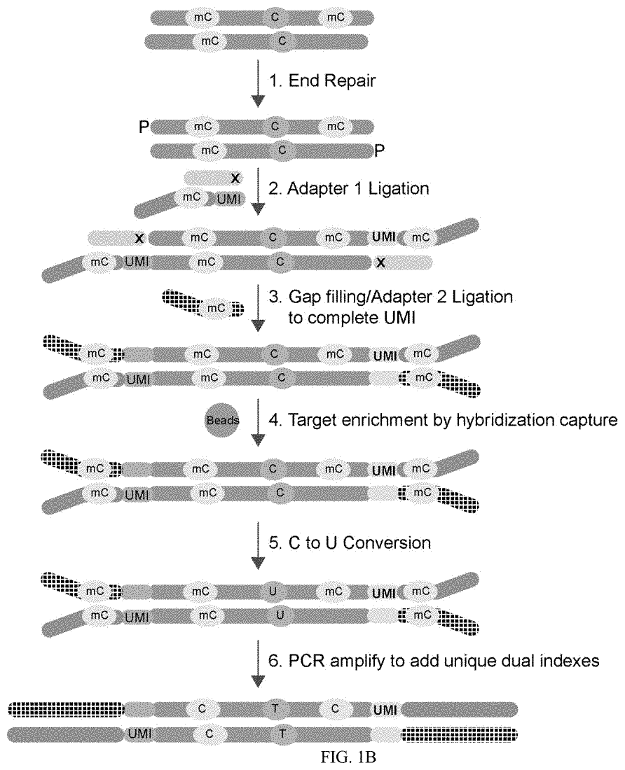

FIG. 1A illustrates an example for preparing a methyl-seq library suitable for whole genome sequencing. In step 1 the target nucleic acid is end repaired and blunt ends are introduced. The resulting end repaired and blunt ended molecules have 5′ phosphorylated ends with free 3′0H ends. In step 2 adapter 1, comprising a duplex adapter that is blocked on one end is ligated to the 3′ end of the target nucleic acid. For example, the first sequencing adaptor may contain P7 Illumina platform sequences. In one embodiment the ligase used to ligate adapter 1 is a T4 DNA ligase. In another embodiment the ligase used to ligate adapter 1 is a mutant T4 DNA ligase. In a certain embodiment the mutant T4 DNA ligase contains an amino acid substitution at K159, while in other embodiments the mutant T4 DNA ligase contains K159S amino acid substitution. In Step 3 adapter 2 is added through a gap filling and ligation procedure. In step 3 the second sequence adapter is attached to the 5′ ends of the target nucleic acid through a gap fill ligation reaction linking the 3′ ends of the adaptor molecules to the phosphorylated 5′ ends of the target nucleic acids. During the gap-fill ligation, complementary UMI bases are filled in, or polymerized, by TaqIT polymerase using a dNTP mix with dATP, dTTP, dGTP, and methyl-dCTP. In Step 4 the unmethylated cytosine is converted to uracil. Bisulfite treatment or enzymatic treatment may be used to convert the unmethylated cytosine to uracil. Step 5 is an optional PCR step. This optional PCR step may additionally use an uracil compatible DNA polymerase. The optional PCR may be used to add the remaining adapter sequence, sample index, or NGS platform specific sequences necessary for NGS. In some embodiments the full adapter sequence needed for NGS is added through the 2-step ligation process. The adapted target nucleic acid and optionally PCR amplified adapter target nucleic acid, or library, is ready for methylation profiling and sequencing on an appropriate sequencing instrument. In some embodiments the full adapter sequence needed for NGS is added through the 2-step ligation process and the optional PCR is not necessary

FIG. 1B illustrates a method for preparing a methyl-seq library and hybridization capture or enrichment to enrich for certain target regions. In step 1 the target nucleic acid is end repaired to blunt the ends of the nucleic acid. The resulting end repaired and blunt ended molecules have 5′ phosphorylated ends with free 3′-OH ends. In step 2 adapter 1, comprising a duplex adapter that is blocked on one end is ligated to the 3′ end of the target nucleic acid. For example, the first sequencing adaptor may contain P7 Illumina platform sequences. In one embodiment the ligase used to ligate adapter 1 is a T4 DNA ligase. In another embodiment the ligase used to ligate adapter 1 is a mutant T4 DNA ligase, while in certain embodiments the mutant T4 DNA ligase contains a K159S amino acid substitution. In a certain embodiment the mutant T4 DNA ligase contains an amino acid substitution at K159. In Step 3 adapter 2 is added through a gap filling and ligation procedure. In step 3 the second sequence adapter is attached to the 5′ ends of the target nucleic acid through a gap fill ligation reaction linking the 3′ ends of the adaptor molecules to the phosphorylated 5′ ends of the target nucleic acids. During the gap-fill ligation, complementary UMI bases are filled in, or polymerized, by TaqIT polymerase using a dNTP mix with dATP, dTTP, dGTP, and methyl-dCTP. In Step 4 the adapted target sequences are enriched using hybridization capture with a panel for double stranded DNAs. In step 5 the unmethylated cytosine is converted to uracil. Bisulfite treatment or enzymatic treatment may be used to convert the unmethylated cytosine to uracil. Step 6 is an optional PCR. This optional PCR step may additionally use an uracil compatible DNA polymerase. The optional PCR may be used to add the remaining adapter sequence, sample index, or NGS platform specific sequences necessary for NGS. In some embodiments the full adapter sequence needed for NGS is added through the 2-step ligation process. The adapted target nucleic acid and optionally PCR amplified adapter target nucleic acid, or library, is ready for methylation profiling and sequencing on an appropriate sequencing instrument. In some embodiments the full adapter sequence needed for NGS is added through the 2-step ligation process and the optional PCR is not necessary.

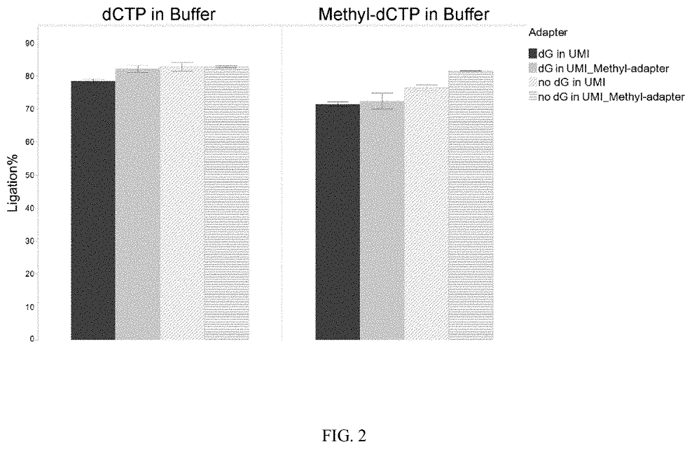

FIG. 2 demonstrates that TaqIT polymerase has a similar incorporation efficiency for incorporating dCTP or methyl-dCTP. dG in the UMI indicates that a dC or methyl-dC will be incorporated onto the opposite strand during the gap filing process. 250 ng 117 bp gBlock was used as insert to test ligation efficiency. 4 types of adapters were examined: Adapters with dG in UMI sequence, adapters without dG in UMI sequence, methylated adapters with dG in UMI sequence, methylated adapters without dG in UMI sequence. In the gap filling/ligation step (FIG. 1A, step 3), buffers with methyl-dCTP, dATP, dTTP, and dGTP were used to test the incorporation efficiency of methyl-dCTP by TaqIT. Buffers with dNTPs (indicated as dCTP in buffer) were used as control.

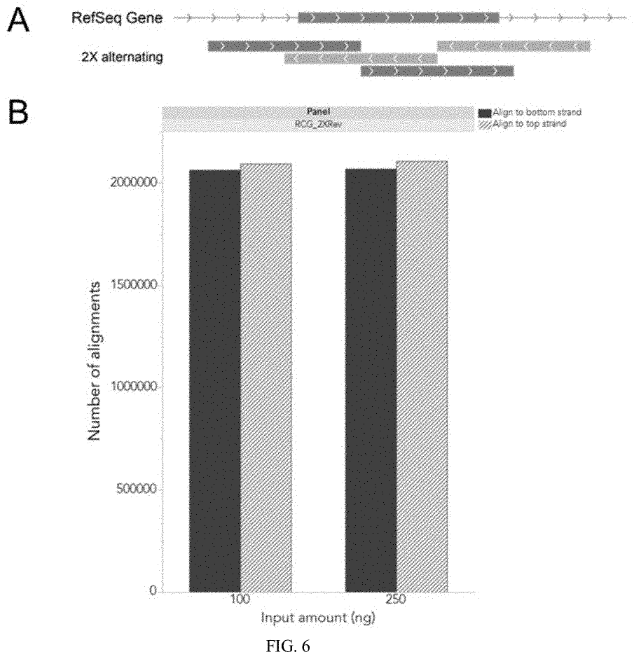

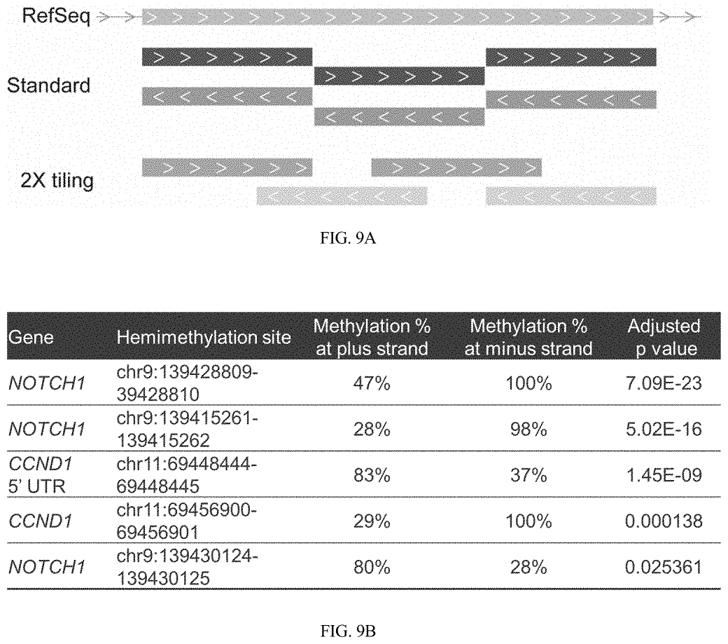

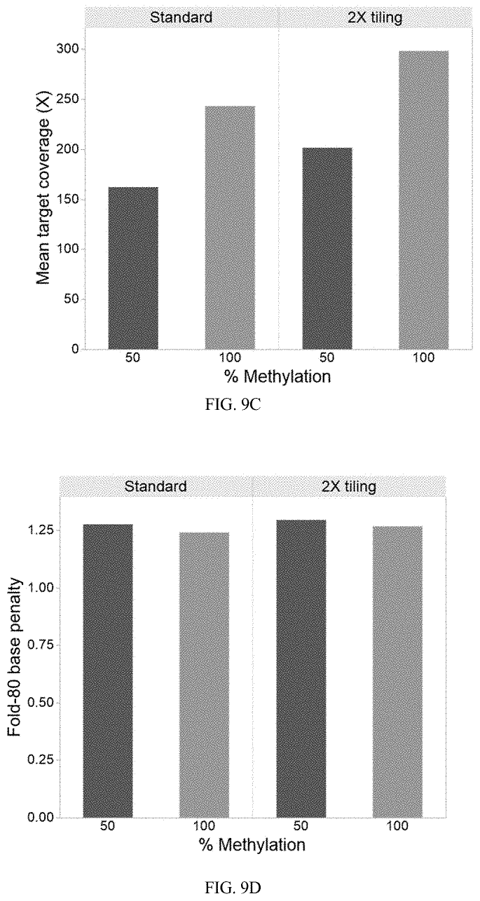

In one embodiment a target enrichment is performed. In certain embodiments amplicon-based enrichment may be used. In certain embodiment hybridization capture enrichment may be used. In another embodiment a 2× alternating panel design for double stranded capture is used. (See FIG. 6A or 9A).

### EXAMPLES

Elements and acts in the examples are intended to illustrate the invention for the sake of simplicity and have not necessarily been rendered according to any particular sequence or embodiment. The example is also intended to establish possession of the invention by the Inventors

### Example 1

Whole Genome Methyl-Seq Library Construction

Target DNA is end repaired and prepared for blunt ligation. A mutant DNA ligase is used to attach 5′ adenylated and methylated adapters to the 3′ end of the target inserts. The complementary portion of the 5′ adapter is blocked to prevent ligation. A gap fill ligation is used to attach Adapter 2 and complementary UMI bases are filled in by TaqIT using a dNTP mix containing dATP, dTTP, dGTP, and methyl-dCTP. Unmethylated cytosine in the target nucleic acid are converted to uracil by bisulfite treatment or enzymatic treatment. PCR amplification of the UMI tagged target sequence is used to introduce unique dual indexes.

FIG. 1A demonstrates one embodiment of the workflow used to add UMI adapters to target nucleic acid, conversion of the unmethylated cytosine, and PCR amplification to add unique dual indexes and appropriate NGS platform specific adapter sequences. The prepared target sequence is then sequenced on the appropriate NGS platform. Following sequencing the sequence is compared to a reference sequence to determine a methylation profile.

1-250 ng fragmented DNA is subjected to an end-repair reaction using T4 Polynucleotide Kinase and T4 DNA Polymerase at 20 ° C. for 30 min. Following end-repair, the first sequencing adaptor (P7 for Illumina platforms) is attached to the 3′ end of insert DNA via blunt ligation using a mutant T4 DNA ligase K159S at 20 ° C. for 15 min. The mutant ligase is then heat inactivated at 65° C. for 15 min. The second sequencing adaptor is then attached to the 5′ ends of biological inserts through a gap fill ligation reaction at 65° C. for 30 min. During the gap-fill ligation, complementary UMI bases are polymerized (filled in) by TaqIT using a dNTP mix with dATP, dTTP, dGTP and methyl-dCTP. Taq ligase is used to ligate the nick between the insert and TaqIT-extended adaptor. Following the second ligation, unmethylated cytosine is converted to uracil by bisulfite reaction or enzymatic treatment using the manufacturer's protocol. The newly constructed library molecules can then be PCR amplified with an uracil compatible DNA polymerase to add sample barcodes. The resultant library is ready for whole genome bisulfite sequencing on Illumina platforms.

TABLE 1

| | | | | | | Ave. |
|---|---|---|---|---|---|---|
| Input | | | Conver- | | Ave. | Library |
| amount | Sample | Work- | sion | PCR | Yield | size |
| (ng) | Type | flow | method | cycles | (ng) | (bp) |
| 1 | NA12878 | Hawkeye | Bisulfite | 16 | 77 | 330.5 |
| 10 | Methylated | Hawkeye | Bisulfite | 14 | 833 | 424.3 |
| | HCT116 | | | | | |
| 10 | NA12878 | Hawkeye | Bisulfite | 14 | 755 | 382.6 |
| 25 | NA12878 | Hawkeye | Bisulfite | 14 | 798 | 358.8 |
| 100 | NA12878 | Hawkeye | Bisulfite | 10 | 199 | 366.0 |
| 250 | NA12878 | Hawkeye | Bisulfite | 10 | 1179 | 372.5 |
| 10 | NA12878 | Hawkeye | Enzyme | 8 | 95 | 390.5 |
| 100 | NA12878 | Hawkeye | Enzyme | 5 | 193 | 378.0 |
| 10 | Methylated | NEB | Enzyme | 8 | 46 | 403.7 |
| | HCT116 | | | | | |
| 10 | NA12878 | NEB | Enzyme | 8 | 52 | 347.0 |

Table 1 shows WGBS libraries prepared from sheared human genomic DNA (NA12878) with varied target nucleic acid input amounts (Nucleic acid input ranging from 1-250 ng). Unmethylated cytosine were converted by EZ DNA methylation-Gold kit (Zymo) (Bisulfite Conversion method) or NEBNext® Enzymatic Methyl-seq Conversion Module (NEB) (Enzyme Conversion Method). PCR cycles were optimized to achieve library yield sufficient for Illumina sequencing. Table 1 shows that adequate library yield and average library size is adequate from 1 ng to 250 ng input nucleic acid amounts. Additionally, Table 1 demonstrates that appropriate Library Size (as measured in base pair (bp)) is obtained.

### Example 2

Targeted Methyl-Seq Library Construction

DNA is end repaired and prepared for blunt ligation. A mutant DNA ligase is used to attached 5′ adenylated and methylated adapters to the 3′ end of the target inserts. The complementary portion of the 5′ adapter is blocked to prevent ligation. A gap fill ligation is used to attached Adapter 2 and complementary UMI bases are filled in by TaqIT using a dNTP mix containing dATP, dTTP, dGTP, and methyl-dCTP. Target regions are captured and enriched by hybridization capture methods. The hybridization capture panel utilizes a 2× alternating panel design for double stranded capture. (see FIG. 6). Following hybridization capture unmethylated cytosine in the target nucleic acid are converted to uracil by bisulfite treatment or enzymatic treatment. PCR amplification of the UMI tagged target sequence is used to introduce unique dual indexes.

FIG. 1B demonstrates one embodiment of the workflow used to add UMI adapters to target nucleic acid, hybridization capture of target regions, conversion of the unmethylated cytosine, and PCR amplification to add unique dual indexes and appropriate NGS platform specific adapters. The prepared target sequence is then sequenced on the appropriate NGS platform.

### Example 3

Detection of Methylation by WGBS Using Bisulfite Conversion of Unmethylated Cytosine

10 ng human genomic DNA (EpiScope Methylated HCT116 and NA12878) was mixed with 5% of unmethylated lambda DNA and sheared to 150 bp using the Covaris S2 instrument. EpiScope Methylated HCT116 gDNA is genomic DNA purified from human HCT116 cells that is highly methylated using CpG methylase (TaKaRa). Unmethylated lambda DNA was used to monitor the conversion efficiency of bisulfite treatment. Unmethylated cytosine were converted by EZ DNA methylation-Gold kit (Zymo). Libraries were sequenced on an Illumina MiSeq (2×150 base). Bisulfite sequencing data was analyzed by bismark program with default setting.

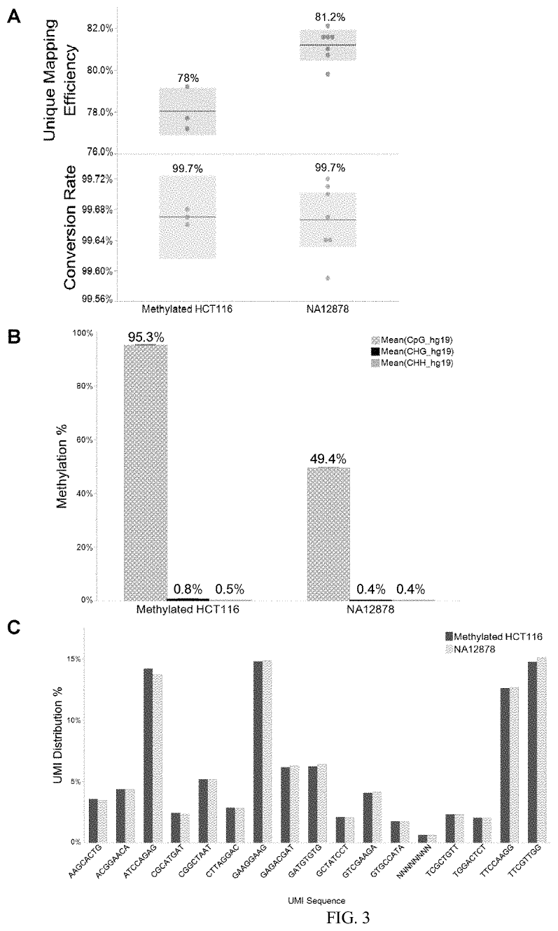

FIG. 3A demonstrates a 99.7% Cytosine to Uracil conversion rate and ˜80% unique mapping efficiency was obtained from both sample types. FIG. 3B shows that methylation levels for methylated HCT116 are 96.3%, 0.8%, and 0.5% in CpG, CHH and CHG contexts. Methylation levels for NA12878 are 49.5%, 0.4%, and 0.4% in CpG, CHH and CHG contexts. FIG. 3C shows the distribution frequency of the 16 rationally designed UMIs and the fixed sequence used. Unmapped reads were measured as NNNNNNNN. The plot of UMI distribution shows that all rationally designed adapter UMIs ligate efficiently.

### Example 4

Detection of Methylation Using Enzymatic Conversion of Unmethylated Cytosine

10 and 100 ng human genomic DNA (NA12878) was mixed with 1% of unmethylated lambda DNA and sheared to 150 bp using the Covaris S2 instrument. Unmethylated cytosine were converted by NEBNext® Enzymatic Methyl-seq Conversion Module. Libraries were sequenced on an Illumina MiSeq (2×150 base). Enzymatic methyl-seq data was analyzed by bismark program with default setting.

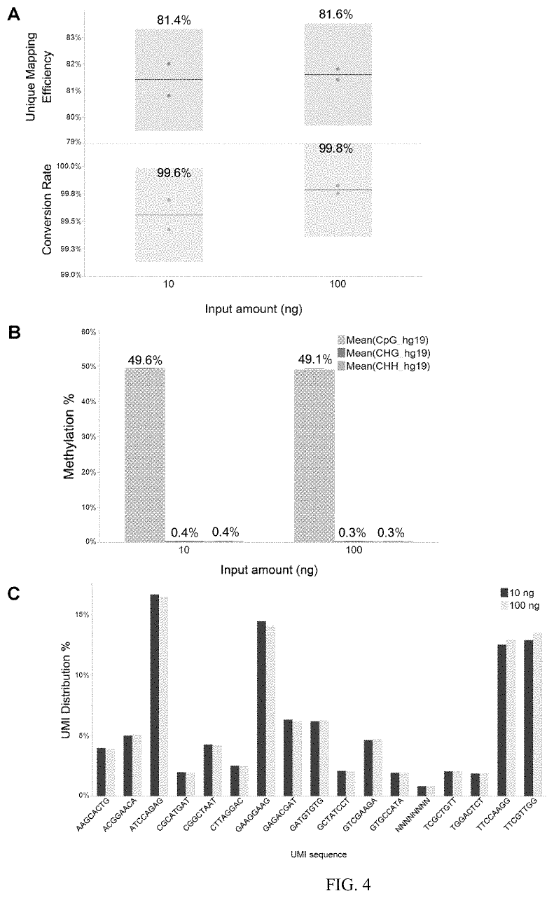

FIG. 4A shows 99.7% Cytosine to Uracil conversion rate and ˜81% unique mapping efficiency were obtained. FIG. 4B demonstrates methylation levels for NA12878 are ˜49%, 0.4%, and 0.4% in CpG, CHH and CHG contexts. FIG. 4C shows the distribution frequency of the 16 rationally designed UMIs and the fixed sequence used. Unmapped reads were measured as The plot of UMI distribution shows that all rationally designed adapter UMIs ligate efficiently

### Example 5

Detection of Methylation and Targeted Enrichment

Targeted methyl-seq libraries were prepared from 25, 50, 100 and 250 ng sheared human gDNA (NA12878) using the workflow (FIG. 1B) and enriched using the Integrated DNA Technologies, Inc., xGen AML panel. Unmethylated cytosine was converted to uracil using the EZ DNA methylation-Gold kit (Zymo).

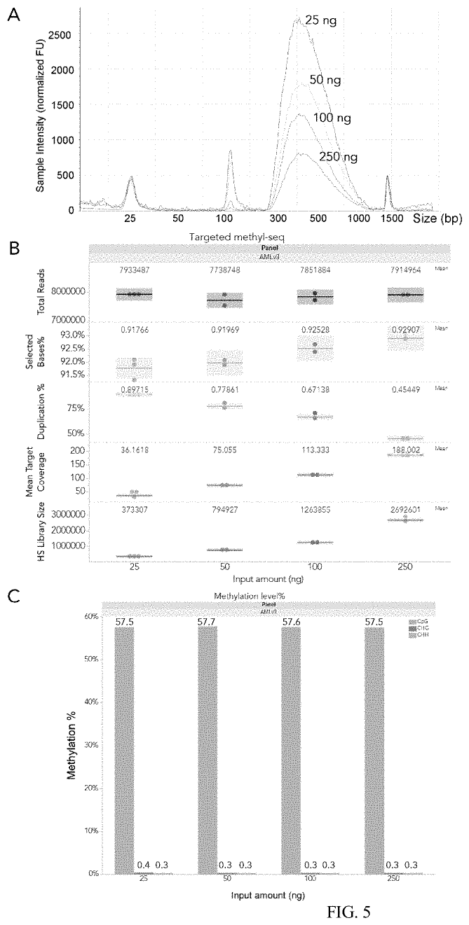

FIG. 5A shows final library traces that were examined on the Agilent TapeStation. FIG. 5B shows targeted methyl-seq libraries that were prepared from 250 ng methylated HCT 116 and NA12878 gDNAs and sequenced on an Illumina MiSeq (2×150 base). Targeted methyl-seq data was analyzed by bismark program and Picard toolkit with default settings. 91.7˜92.9% selected bases on the target regions and 36-188× mean target coverage were obtained, suggesting that the methylation events occur within the target regions can be identified with higher sensitivity. FIG. 5C shows methylation levels for NA12878 gDNA are ˜58%, 0.3%, and 0.3% in CpG, CHH and CHG contexts.

### Example 6

Libraries were generated from 10 ng of methylation controls with 0, 5, 10, 25, 50, 100% of methylation (EpigenDx) as described in Example 1. Unmethylated cytosine were converted by EZ DNA methylation-Gold kit (Zymo). Libraries were sequenced on an Illumina NextSeq (2×150 base).

Alignment and methylation analyses were performed using Bismark (v0.22.3) and

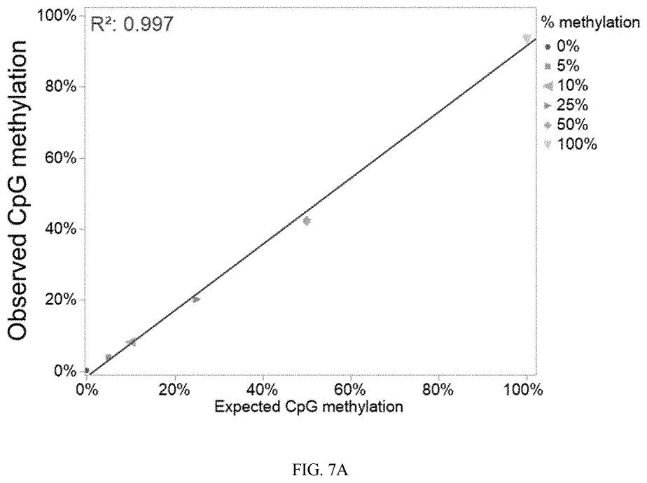

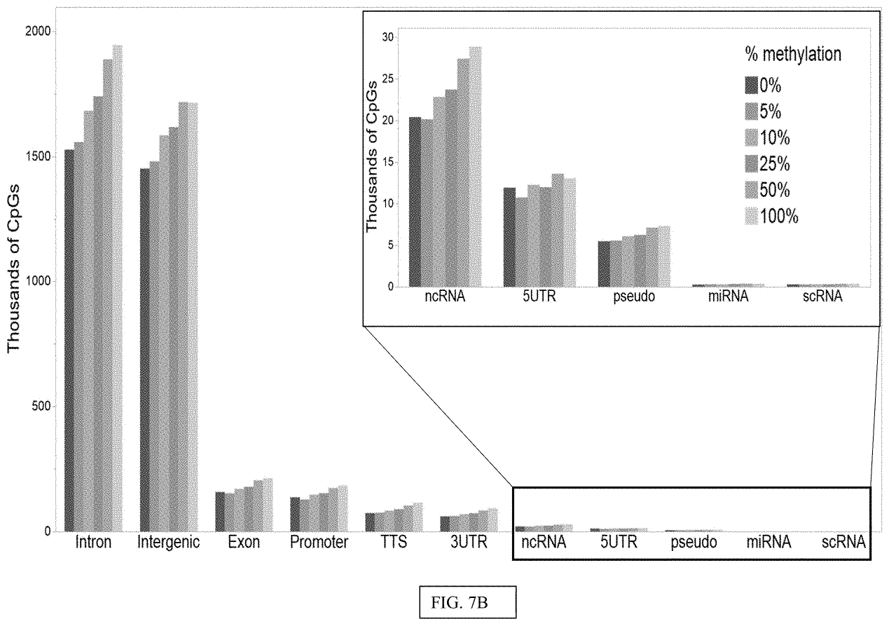

Picard (v2.18.9) and genomic features were annotated using Homer (Hypergeometric Optimization of Motif EnRichement) for motif discovery. FIG. 7A shows the high correlation between expected and observed methylation levels. FIG. 7B identifies a wide range of genomic features, including transcriptional regulatory regions, using Homer after sequencing to 36 M reads. FIG. 7B shows the number of CpG sites that are identified on the Y axis and the annotated motif/region on the x axis. The figure shows the workflow can cover/identify various genomic features with no/little bias for the inputs with various methylation levels.

### Example 7

10 ng of cfDNA from healthy individuals and individuals with lung cancer were put into library preparation as described in Example 1. Unmethylated cytosine were converted by EZ DNA methylation-Gold kit (Zymo). Libraries were sequenced on an Illumina NextSeq (2×150 base).

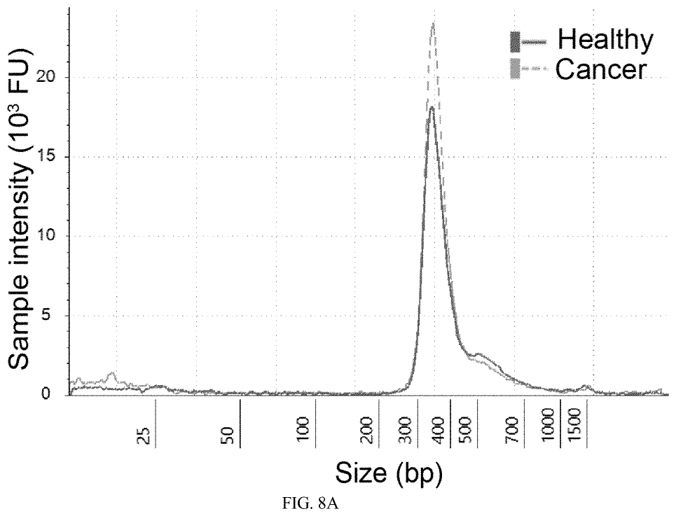

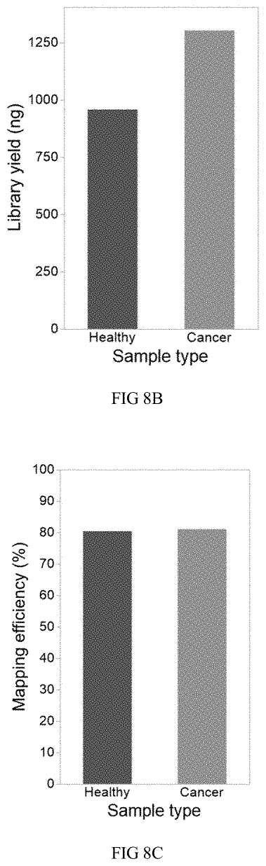

Alignment and methylation analyses were performed using bismark program with default setting. FIG. 8(A) shows a representative electropherograms from libraries using the described methylation workflow. FIG. 8(B) demonstrates the workflow provides >1 μg library yield from 10 ng of cfDNA. FIG. 8(C) shows that ˜80% unique mapping efficiency was obtained from both healthy and cancer samples.

### Example 8

Alternating design in targeted methyl-seq captures both strands for hemimethylation analysis.

Targeted methyl-seq libraries were prepared from sheared, 100 ng of 50% and 100% methylation controls (EpigenDx) using the workflow (FIG. 1B) and enriched using two designs of 130 kb, custom panel to target CpG islands, shores and shelves within oncogenes. For the first standard panel design, we used IDT xGen v2 pipeline with end-to-end algorithm. The initial output probe design is only for one strand of DNA. To target both DNA strands, we added and reverse-complemented the probes to target the other strand (FIG. 9A). For the second 2× tiling design, we used IDT xGen v2 pipeline with 2× tiling algorithm. To target both DNA strands, we swapped the targeted strands for every other probe (FIG. 9A). Unmethylated cytosine were converted by EZ DNA methylation-Gold kit (Zymo). Libraries were sequenced on an Illumina NextSeq (2×150 base). Alignment and methylation analyses were performed using Bismark (v0.22.3) and Picard (v2.18.9). DNA strands were captured with ˜70% on-target rate. FIG. 9B shows Hemimethylation sites were identified by applying Fisher's exact test, then adjusting all p-values using the Benjamini-Hochberg procedure with a false-discovery error rate of 0.05. FIG. 9C shows 150-300× of mean targeted coverage was observed after downsampling to 16 M reads. FIG. 9D demonstrates that both panel designs provide high capture uniformity.

All references, including publications, patent applications, and patents, cited herein are incorporated by reference to the same extent as if each reference were individually and specifically indicated to be incorporated by reference and were set forth in its entirety herein.

The use of terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but no limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

Valouev et al. Methods of preparing dual-indexed DNA libraries for bisulfite conversion sequencing. US Patent Application: US20180044731A1

Gai, W. and K. Sun, Epigenetic Biomarkers in Cell-Free DNA and Applications in Liquid Biopsy. Genes (Basel), 2019. 10(1).

Liu, Y., et al., Bisulfite-free direct detection of 5-methylcytosine and 5-hydroxymethylcytosine at base resolution. Nat Biotechnol, 2019. 37(4):

Moss, J., et al., Comprehensive human cell-type methylation atlas reveals origins of circulating cell-free DNA in health and disease. Nat Commun, 2018. 9(1): p. 5068.

Schutsky, E.K., et al., APOBEC3A efficiently deaminates methylated, but not TET-oxidized, cytosine bases in DNA. Nucleic Acids Res, 2017. 45(13): p. 7655-7665.

What is claimed is:

1. A method of determining a methylation profile of a target nucleic acid comprising:
a) obtaining the target nucleic acid;
b) ligating a first adapter to the 3′ end of the target nucleic acid with a first ligase;
c) ligating a second adapter to the 5′ end of the target nucleic acid with a second ligase to generate an adapter-target-adapter complex;
d) converting unmethylated cytosine to uracil in the adapter-target-adapter complex to generate a converted target;
e) optionally PCR amplifying the converted target;
f) sequencing the converted target;
g) comparing the sequence of the converted target to a reference sequence to determine the methylation profile of the target nucleic acid.

2. The method of claim 1, wherein the target nucleic acid molecules are DNA

3. The method of claim 2, wherein the DNA is whole genomic DNA, cfDNA, or FFPE DNA.

4. The method of claim 1, wherein the first ligase is a T4 DNA ligase.

5. The method of claim 4, wherein the T4 DNA ligase is a mutant ligase is.

6. The method of claim 5, wherein the mutant ligase contains an amino acid substitution at K159.

7. The method of claim 1, wherein the first adapter or second adapter contains a unique molecular identifier sequence.

8. The method of claim 1, wherein the first adapter and second adapter contain a unique molecular identifier sequence.

9. The method of claim 1, wherein the converting unmethylated cytosine to uracil comprises treatment with bisulfite.

10. The method of claim 1, wherein the converting unmethylated cytosine to uracil comprises treatment with a cytidine deaminase.

11. The method of claim 1, wherein the adapters comprise a universal priming site.

12. The method of claim 1, wherein the adapter-target-adapter complex is enriched by hybridization capture.

13. The method of claim 1, wherein steps a) through g) are performed in order.
